(19) United States
(12) Patent Application Publication
(10) Pub. No.: US 2015/0157278 A1
(43) Pub. Date: Jun. 11, 2015

(54) ELECTRONIC DEVICE, METHOD, AND STORAGE MEDIUM

(71) Applicant: KABUSHIKI KAISHA TOSHIBA, Tokyo (JP)

(72) Inventors: Sawa FUKE, Kawasaki (JP); Kanako Nakayama, Tokyo (JP); Junya Takakura, Kawasaki (JP)

(21) Appl. No.: 14/564,752

(22) Filed: Dec. 9, 2014

(30) Foreign Application Priority Data
Dec. 10, 2013 (JP) 2013-255272

(51) Int. Cl. A61B 5/00; G08C 17/02; G06T 11/20

(52) U.S. Cl. CPC A61B 5/743; G06T 11/206; G08C 17/02

(57) ABSTRACT

According to one embodiment, an electronic device is configured to control a sensor device executing applications to measure different biomedical data values. The electronic device includes a display controller and a transmitter. The display controller displays a first image for designating an application to be executed by the sensor device, designating an execution time of the application, and designating an activation condition associated with a biomedical data value measured by the sensor device. The transmitter transmits, to the sensor device, the designated application, the designated execution time and the designated activation condition.

### CROSS-REFERENCE TO RELATED APPLICATIONS

This application is based upon and claims the benefit of priority from Japanese Patent Application No. 2013-255272, filed Dec. 10, 2013, the entire contents of which are incorporated herein by reference.

### FIELD

Embodiments described herein relate generally to control of a sensor device for measuring various biomedical data values.

### BACKGROUND

Attaching a sensor device to a human body to measure biomedical data values for health management has been devised recently. The sensor device incorporates a plurality of sensors, and can measure various biomedical data values by analyzing the output of each sensor or combinations of the outputs of the sensors.

Thus, a sensor device incorporating a plurality of sensors and a microcomputer with programs installed therein for performing control and analysis can measure various biomedical data values by selecting a sensor and a program. The type of necessary biomedical data and/or a measurement period of time differs between users. Further, when a plurality of biomedical data values are processed by a microcomputer installed in the device, there are limitations on the combinations of simultaneously usable processings in view of the memory capacity or processing amount. Further, when the device is powered by a battery, the operating time of the device decreases if the number of biomedical data values to be measured increases. Therefore, there is a demand for controlling the operation of the sensor device to enable the device to perform measurement of each biomedical data value necessary for each user within a necessary time period.

### BRIEF DESCRIPTION OF THE DRAWINGS

FIG. 1 is an outline view showing an entire configuration example of an embodiment.

FIG. 2 is an exemplary plan view showing the reverse side (that is to be attached to a living body) of the biomedical sensor device according to the embodiment.

FIG. 3 is a block diagram showing a circuit configuration example of the biomedical sensor device of the embodiment.

FIG. 4 is a block diagram showing a circuit configuration example of a tablet as an electronic device of the embodiment.

FIG. 5 is a flowchart showing an example of a scenario registration operation of the tablet of the embodiment.

FIG. 6 is a flowchart showing in detail an example of the application display processing shown in FIG. 5.

FIG. 7 shows an example of a user registration screen image displayed on the tablet of the embodiment.

FIG. 8 shows an example of an application select initial screen image displayed on the tablet of the embodiment.

FIG. 9 shows an example of an index select screen image displayed. on the tablet of the embodiment.

FIG. 10 shows an example of a categorized application display screen image displayed on the tablet of the embodiment.

FIG. 11 shows a screen image example of the tablet of the embodiment, Le an application display screen image for a size/color corresponding to a type.

FIG. 12 shows a screen image example of the tablet of the embodiment, i.e., a display screen image of displaying an application that was determined abnormal last time, and an application associated therewith.

FIG. 13 shows a screen image example of the tablet of the embodiment, i.e., a display screen image of displaying an application downloaded from a server.

FIG. 14 shows an alert screen image example displayed on the tablet of the embodiment.

FIG. 15 shows an example of a scenario registered using the tablet of the embodiment.

FIG. 16 shows an example of registered content of an autonomic nerve analyzing application employed in the embodiment.

FIG. 17 shows an example of registered content of an exercise volume calculating application employed in the embodiment.

FIG. 18 shows an example of registered content of a body temperature measuring application employed in the embodiment.

FIG. 19 shows an example of registered content of a fall predicting application employed in the embodiment.

### DETAILED DESCRIPTION

In general, according to one embodiment, an electronic device is configured to control a sensor device executing applications to measure different biomedical data values. The electronic device includes a display controller and a transmitter. The display controller displays a first image for designating an application to be executed by the sensor device, designating an execution time of the application, and designating an activation condition associated with a biomedical data value measured by the sensor device. The transmitter transmits, to the sensor device, the designated application, the designated execution time and the designated activation condition.

FIG. 1 shows an example of a health management system including an electronic device, according to an embodiment, This system includes a biomedical sensor device 10 attached to a living body (such as a human body or animal), electronic device 12, such as a tablet, a PC or smartphone, Internet 14 and server 16. A medical agency, such as a hospital, an enterprise health management association, an elderly care association, an education agency, etc., are supposed to be administrators of the health management system. Health management is realized by attaching the biomedical sensor device 10 to patients or workers, and permitting the administrators to always monitor their biomedical data via the electronic device 12 and Internet 14 for early detection of physical abnormalities.

The biomedical sensor device 10 is compact, light and thin, and is powered by a battery (e.g., a built-in rechargeable battery). To enable biomedical data values to be always measured, the biomedical sensor device 10 is attached to a human body by means of, for example, adhesive tape. However, attachment to the human body is not limited to that using an adhesive material, but may be attachment via a wristband or earphones. Alternatively, the sensor device 10 may be built in another component, such as cloth or shoes.

The biomedical sensor device 10 has a function of simultaneously measuring a plurality of values associated with biomedical data, such as a pulse wave, electrocardiographic wave, temperature, acceleration of gravity, blood oxygen level, etc., and wirelessly transmitting the measurement result to electronic device 12. A sensor may be a microphone to pick up snore. The biomedical sensor device 10 also has a function of wirelessly receiving, for example, control signals from the electronic device 12.

The electronic device 12 also can monitor biomedical data. However, since the electronic device 12 is degraded in the capacity of processing a large amount of data, compared to the server 16, it is connected to the Internet 14 to upload the biomedical data, sent from the biomedical sensor device 10, to the server 16 on the Internet 14, and to download data from the server 16 and send the same to the biomedical sensor device 10. The connection between the electronic device 12 and Internet 14 is not limited to wireless connection, but may be wired one. Further, the Internet 14 and server 16 are dispensable. The electronic device 12 may include the function of the server 16, and the health management system may be constituted of the biomedical sensor device 10 and electronic device 12. Similarly, the electronic device 12 can be dispensed with. If the biomedical sensor device 10 is connectable to the Internet 14, the function of the electronic device 12 may be imparted to the server 16, and the health management system may be constituted of the biomedical sensor device 10, Internet 14 and server 16.

The biomedical sensor device 10 has a plurality of sensors so as to realize simultaneous measurement of a plurality of biomedical data values. However, since the analog front ends of the sensors have different specifications, simultaneous pursuit of flexibility and high performance is required for the sensor device, which may involve an increase in size. The embodiment, however, employs quasi SoC technique to integrate a plurality of analog front ends, a CPU, etc. on a chip, thereby realizing a square sensor module with each side of several millimeters. The quasi SoC technique is a technique of integrating components on a wafer to simultaneously establish downsizing corresponding to SoC and design freedom corresponding to SiP. By connecting a few peripheral components, such as an antenna and a battery, to the module, the biomedical sensor device 10, which is small, light (about 10 and several grams) and thin (about several millimeters), is realized. Although in the embodiment, downsizing of the biomedical sensor device 10 is realized using quasi SoC technique, it may be realized using, for example, LSI.

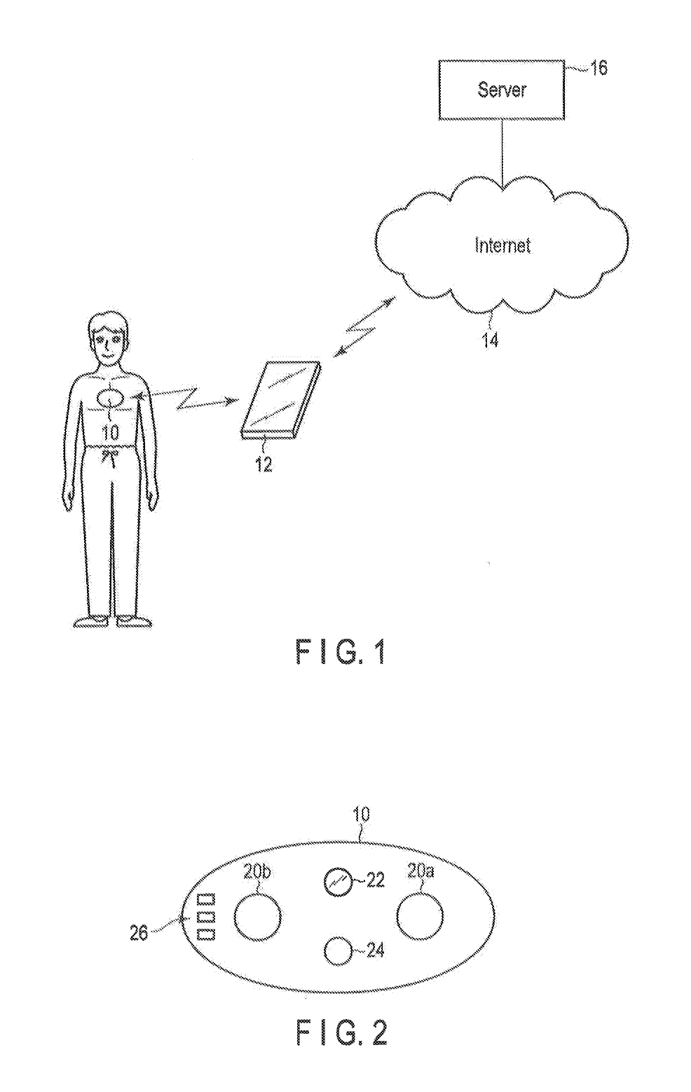

The biomedical sensor device 10 has an elliptic shape with a major axis of, for example, about several cm, and has a surface attached to a human body and provided with an electrocardiograph electrode (R) 20a, electrocardiograph electrode (L) 20b, photoelectric unit 22, temperature sensor 24 and charge terminal 26, as is shown in FIG. 2. The electrocardiograph electrodes 20a and 20b is preferably positioned at right and left portions of a heart, respectively, and are therefore arranged along the major axis with an interval. The photoelectric unit 22 is configured to optically detect a pulse wave, and has a light-transmitting transparent window at its front surface.

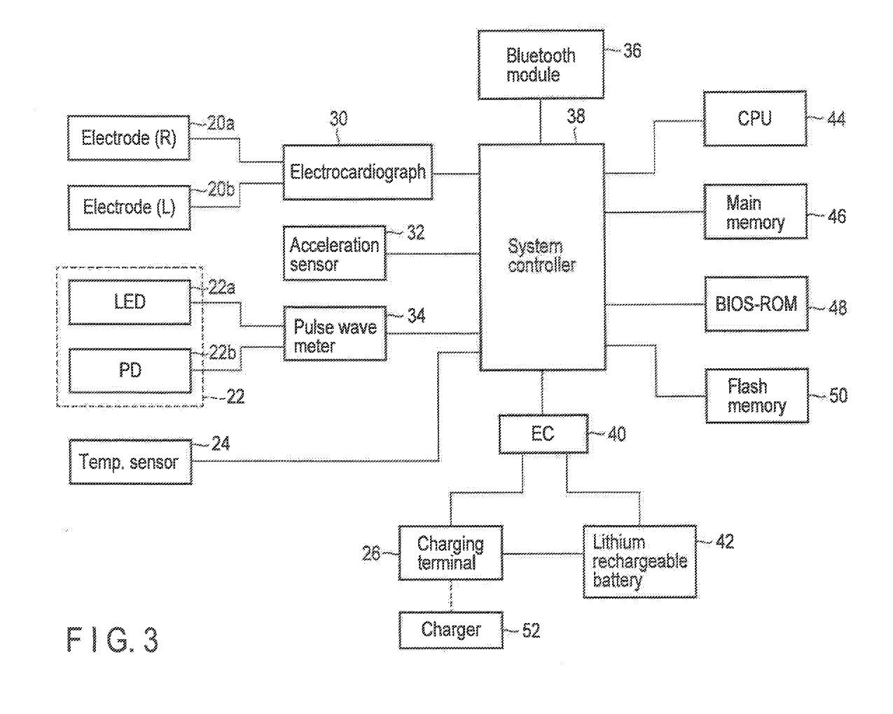

FIG. 3 is a block diagram showing a circuit configuration example of the biomedical sensor device 10. In addition to the above-mentioned electrocardiograph electrodes 20a and 20b, photoelectric unit 22, temperature sensor 24 and charge terminal 26, the biomedical sensor device 10 incorporates an electrocardiograph 30, acceleration sensor 32, pulse wave meter 34, Bluetooth (trademark) module 36, system controller 38, embedded controller (EC) 40, lithium rechargeable battery 42, CPU 44, main memory 46, BIOS. ROM 48, flash memory 50, etc.

The electrocardiograph electrode (R) 20a and electrocardiograph electrode (L) 20b are connected to the electrocardiograph 30 as an analog front end for electrocardiogram. The electrocardiograph 30 obtains an electrocardiogram by analyzing a time-sequence signal that is obtained by sampling potential differences between the electrocardiograph electrode (R) 20a. and electrocardiograph electrode (L) 20b, The electrocardiograph 30 also obtains a cardiac rate from the electrocardiogram, and obtains an R-R interval (RRI) as the interval between two R waves corresponding to subsequent two heart beats.

The photoelectric unit 22 is configured to detect a pulse wave (plethsymogram), and has a light emitting element (e.g., a blue LED) 22a and a photodiode (PD) 22b as a light receiving element. A transparent window is provided at the front surface of the photoelectric unit 22, through which light from the blue LED 22a is applied to a skin surface, and through which light reflected from the skin surface enters the PD 22b. The blue LED 22a and PD 22b are connected to the pulse wave meter 34 as an analog front end for pulse waves. The pulse wave meter 34 detects variation in the level of reflected light due to variation in the amount of blood in capillary vessel, and analyzes the detection signal to obtain a pulse wave and then the number of pulses.

The electrocardiograph 30, acceleration sensor 32, pulse wave meter 34 and temperature sensor 24 are connected to the system controller 38. The temperature sensor 24 measures the temperature of the surface of the human body, and the acceleration sensor 32 measures motion of the human body.

The CPU 44 is a processor configured to control the operation of each of the modules and components of the biomedical sensor device 10. As described above, by analyzing the output of each sensor of the biomedical sensor device 10, or analyzing a combination of the outputs of sensors of the biomedical sensor device 10, values indicative of various situations of a living body can be measured. Which sensor outputs are used, how sensor outputs are combined, and what biomedical data value(s) is measured are defined as an application program (hereinafter, referred to simply as an application). For instance, application examples include a blood pressure measuring application, an autonomic nerve analyzing application, a sleep analyzing application, an exercise volume calculating application, a body temperature measuring application, a falling prediction application, etc. The exercise volume calculating application and the falling prediction application are used to measure necessary data values based on the output of the acceleration sensor 32.

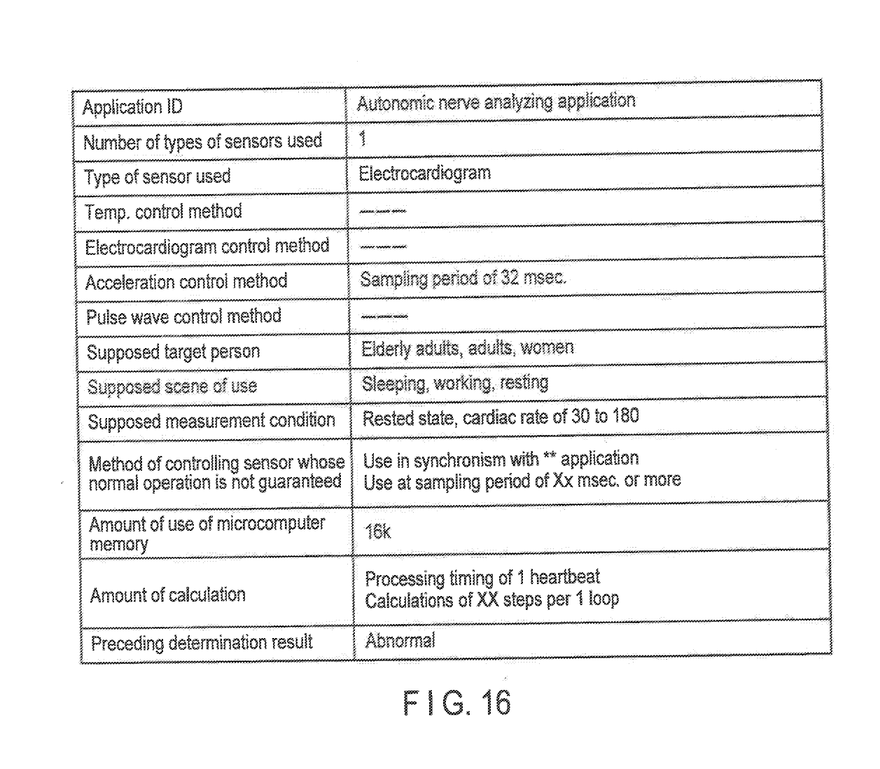

These applications are beforehand prepared by, for example, the manufacture of the biomedical sensor device 10 or the administrator of the health management system, and are registered in the server 16 (in the case where the system does not include the server 16, they are registered in the electronic device 12). For particulars, the applications will be described later with reference to FIG. 16. The electronic device 12 can control the operation of the biomedical sensor device 10 by installing, in the biomedical sensor device 10 when necessary, applications downloaded from the server 16 or applications incorporated in the electronic device 12 itself.

Blood pressure is detected based on a pulse wave transit time (PWTT) associated with peaks (R-wave peaks) of an electrocardiogram waveform and peaks of the pulse wave. The pulse wave transit time indicates the time interval be the time when an R wave in an electrocardiogram has appeared and the time when a peripheral pulse wave has appeared. The pulse wave transit time is reversely proportional to the blood pressure. Accordingly, variation in blood pressure can be determined from the pulse wave transit time (PWTT). Further, to predict variation in blood pressure, not only the pulse wave transit time, but also a feature amount, such as the amplitude or area of the waveform of the pulse wave, or the amplitude of an acceleration pulse wave, may be utilized as a variable. In blood pressure measurement, an initial value may be predetermined. For instance, a user blood pressure measured by a standard blood-pressure measure, a pulse wave transit time or another feature amount at this time, may be stored as an initial value in the flash memory 50. Using variation in blood pressure obtained by a current pulse wave transit time (PWTT) and the feature amount, and the initial values (indicative of the relationship between the blood pressure and the pulse wave transmit time or the feature amount), the current blood pressure of the user can be determined. Alternatively, by preparing standard data indicative of the relationship between the blood pressure and the pulse wave transit time or the feature amount, instead of inputting, as initial values, the user blood pressure measured by the standard blood-pressure measure and the pulse wave transit time or the feature amount at this time, the current blood pressure of the user may be determined using the standard data and variation in blood pressure detected from the current pulse wave transit time (PWTT) and the feature amount.

In the autonomic nerve analyzing application, it is estimated which is dominant, the sympathetic nerve or the parasympathetic nerve, based on a heartbeat interval calculated from an electrocardiogram, or on a pulse interval calculated from the pulse wave. This estimation is performed in accordance with a model in which the cardiac rate increases when the sympathetic nerve is active, and decreases when the parasympathetic nerve is active, and based on periodicity of variation in the heartbeat interval. For instance, regarding the variation in the heartbeat interval, a frequency band around 0.1 Hz is associated with both the sympathetic nerve and the parasympathetic nerve, and a frequency band around 0.25 Hz is associated with the parasympathetic nerve. Therefore, by comparing the magnitudes (power levels) of the resultant components of frequency analysis processing, it can be estimated which one of these nerves is dominant.

In the sleep analyzing application, the depth of sleep is measured, based on the estimation result of the sympathetic nerve activation or the parasympathetic nerve activation estimated from the heartbeat interval or the pulse interval by the above-mentioned method, and also based on the amount of motion calculated from an acceleration value.

Although the above-mentioned applications define, for example, the number of types of sensors and the types of sensors needed to measure biomedical data values, they do not define the time period for measuring the biomedical data values, i.e., their execution time periods. The biomedical sensor device 10 is attached to a living body and can always measure biomedical data values. However, there is a biomedical data value that does not have to be always measured. Further, there is a biomedical data value that is meaningless if it is measured when a living body is not in a particular state even during a predefined time period. For instance, biomedical data used to detect an apnea state is obtained only when a living body is in sleep. Such a particular state will be called an application activation condition, i.e., a measurement condition. Therefore, it is necessary to define execution time periods (each indicated by an activation start (time) point and an activation end (time) point) and measurement conditions corresponding to the respective applications. In the embodiment, the execution time period and measurement condition of each application is defined as a scenario. Particulars of the scenario will be described later. The scenario is created by electronic device 12 and set in the biomedical sensor device 10. Alternatively, the scenario may be created on the server 16 side, be downloaded to the electronic device 12, and set in the biomedical sensor device 10. The scenario is stored in the flash memory 50 of the biomedical sensor device 10. The biomedical sensor device 10 with the scenario set therein monitors a current time point, and determines whether the state of the living body satisfies the measurement condition, when the activation start (time) point is reached. If the measurement condition is satisfied, the application defined in the scenario is activated. Scenarios may be created by a number of users, uploaded to the server 16 and collected as a database, so that they can be referred to by any user.

The system controller 38 is a bride device connecting the CPU 44 to each module or component. The system controller 38 is also connected to the Bluetooth module 36, embedded controller (EC) 40, CPU 44, main memory 46, BIOS-ROM 48 and flash memory 50.

The embedded controller 40 is a power management controller for performing power management of the biomedical sensor device 10, and controls charging of a built-in rechargeable battery, such as the lithium rechargeable battery 42. When the charger 52 is attached to the biomedical sensor device 10, the charging terminal 26 is brought into contact with the terminal of charger 52, whereby a charging current is supplied from the charger 52 to the biomedical sensor device 10 via the charging terminal 26 to charge the lithium rechargeable battery 42. Based on the power from the lithium rechargeable battery 42, the embedded controller 40 supplies an operation power to each component. Further, the embedded. controller 40 monitors the charged amount of the lithium rechargeable battery 42 to estimate the time point of power runout (the operation end point of the biomedical sensor device 10).

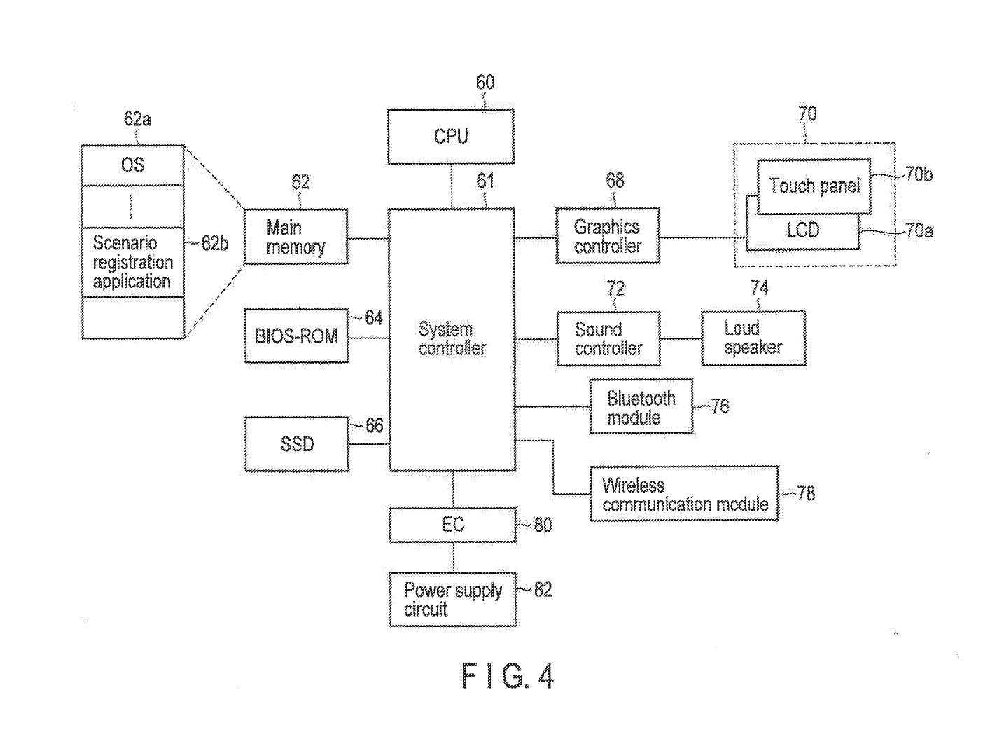

FIG. 4 shows the system configuration of the electronic device 12, Assume here that a tablet terminal is employed as an example of the electronic device 12. The electronic device 12 incorporates a CPU 60, system controller 61, main memory 62, BIOS-ROM 64, solid state drive (SSD) (or hard disk drive (HDD)) 66, graphics controller 68, touch screen display 70, sound controller 72, loud speaker 74, Bluetooth module 76, wireless communication module 78 embedded controller (EC) 80, power supply circuit 82, etc.

The CPU 60 is a processor for controlling the operation of each module or component mounted in the tablet terminal, The CPU 60 executes various types of software loaded from the SSD 66 as a nonvolatile storage device to the main memory 62. The various types of software include operating system (OS) 62a, scenario registration application 62b, etc.

The scenario registration application 62b causes the touch screen display 70 to display scenario registration screen images and sequentially change the screen images in accordance with data input to the screen, thereby enabling the user to select an application, to input an execution time period, and to set a measurement condition, in order to generate/register a scenario. The scenario is stored in the SSD 66, and is also transmitted to the biomedical sensor device 10 and stored in the flash memory 50 of the biomedical sensor device 10.

The CPU 60 also executes basic input/output system (BIOS) stored in the BIOS ROM 64. The BIOS is a program for hardware control.

The system controller 61 is a device for connecting the CPU 60 to each module and component. The system controller 16 includes a memory controller configured to perform access control for the main memory 62. The system controller 61 is connected to the CPU 60, main memory 62, BIOS-ROM 64, SSD 66, graphics controller 68, touch screen display 70, sound controller 72, Bluetooth module 76, wireless communication module 78, embedded controller 80, etc.

The graphics controller 68 controls an LCD 70a used as the display monitor of the electronic device 12. The graphics controller 68 transmits display signals to the LCD 70a under the control of CPU 60. Based on the display signals, the LCD 70a displays screen images (various registration menu screens). A touch panel 70b is provided on the LCD 70a. By touching the screen of touch panel 70b with a finger, various operations can be made. Touch operations include tap and drag operations, etc.

The Bluetooth module 76 is configured to communicate with the Bluetooth module 36 of the biomedical sensor device 10 to receive the biomedical data from the biomedical sensor device 10, and transmit data, such as a scenario created by the electronic device 12, to the biomedical sensor device 10.

The wireless communication module 78 is configured to execute wireless communication, such as wireless LAN communication or 3G mobile communication, or to executes proximity wireless communication, such as near field communication (NFC). The electronic device 12 is connected to the Internet 14 via the wireless communication module 78.

The embedded controller 80 is a one-chip microcomputer including a controller for power management, and is configured to turn on/off the power supply for the electronic device 12 by controlling the power supply circuit 82.

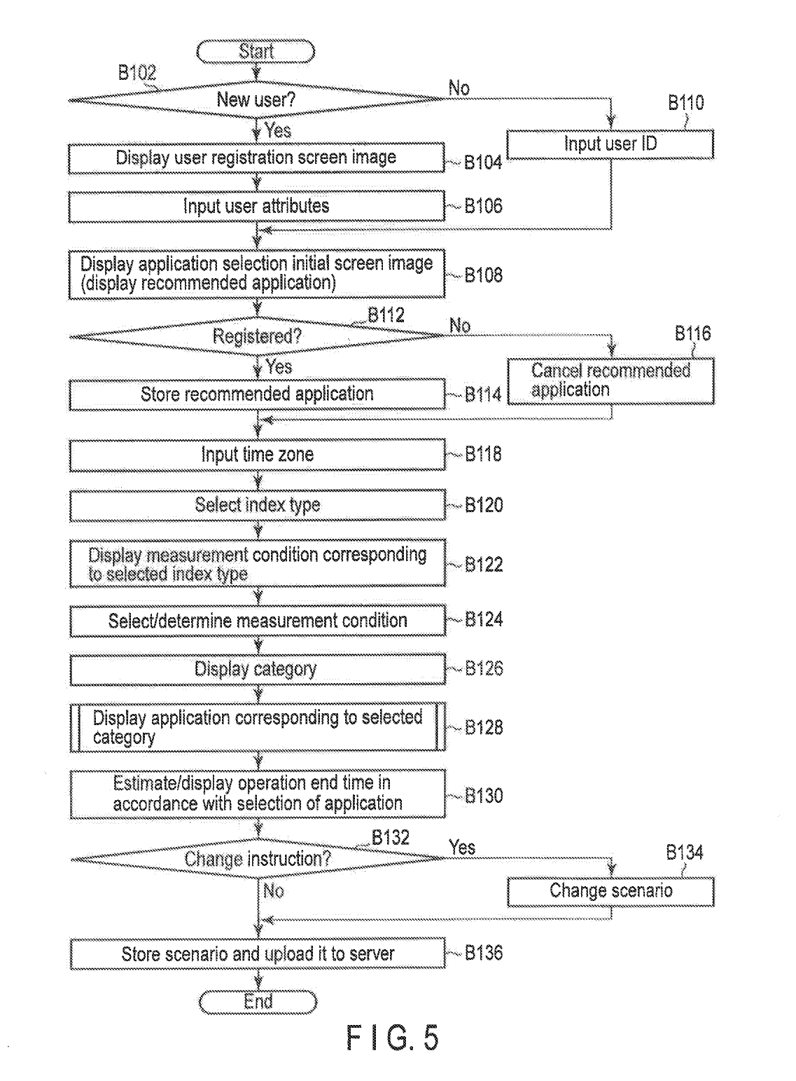

Referring to FIG. 5, a description will be given of a scenario registration operation example of the electronic device 12.

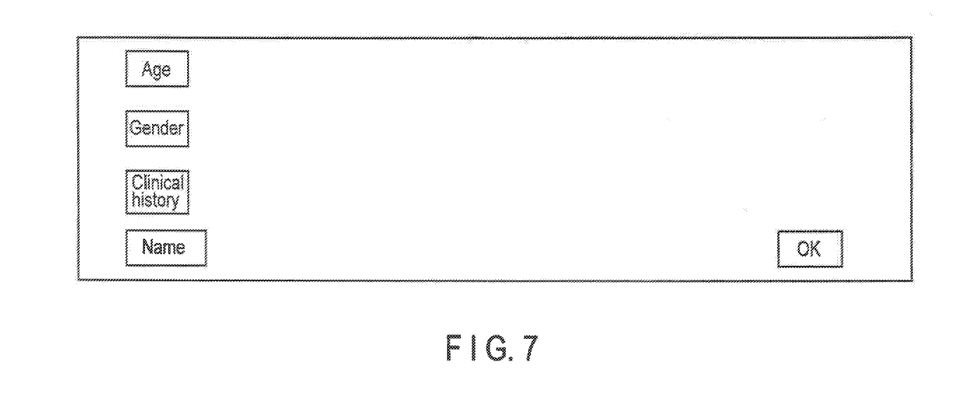

Upon activation of the scenario registration application, a new user registration inquiry screen image (not shown) is displayed in block B102. The inquiry screen image includes an inquiry as to whether the user is a new one or an already existing one. In this case, if the new user is selected, such a new user registration screen image as shown in, for example, FIG. 7 is displayed in block B104. The user registration screen image of FIG. 7 includes boxes associated with user attributes, such as age, gender, clinical history, name, etc., and an OK button. In block B106, a software keyboard is displayed to permit user attributes to be input. Age, gender and clinical history data may be input by permitting the user to tap boxes corresponding thereto and permitting the user to select one of the options displayed in each box.

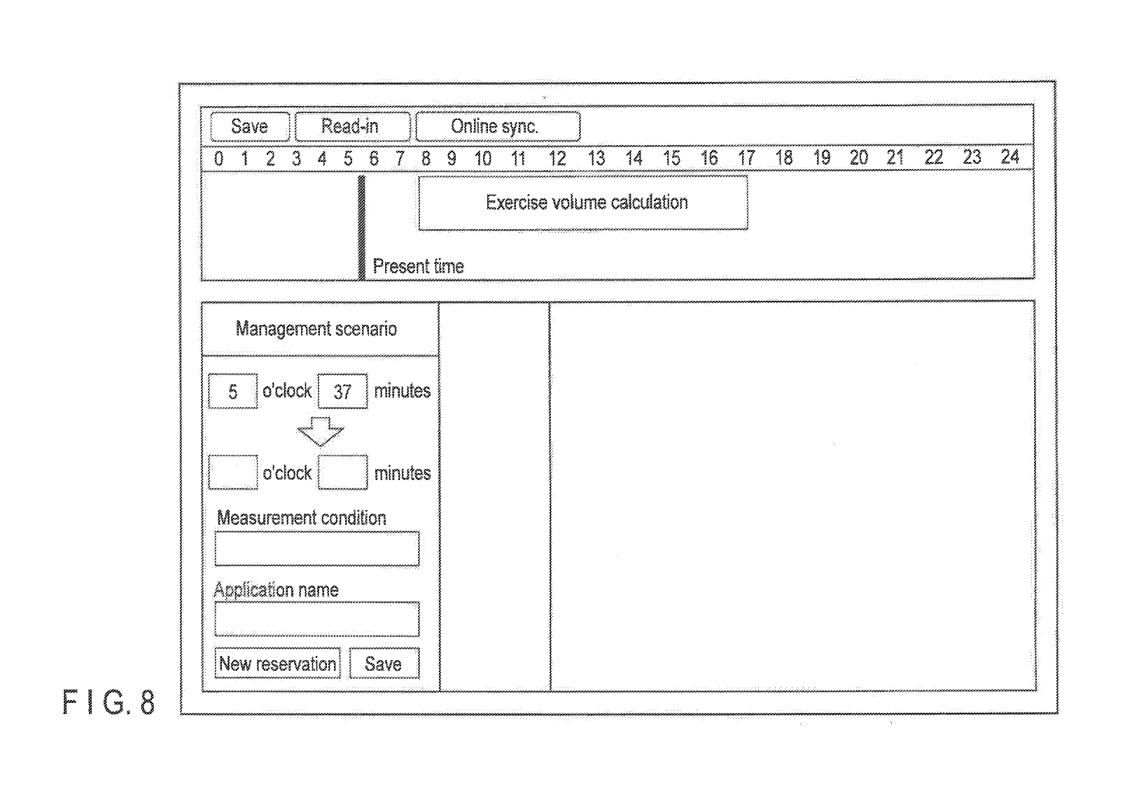

After completing the input operation and tapping the OK button, user ID is reported, In block B108, such an application selection initial screen image as shown in FIG. 8 is displayed. If the already existing user has been selected from the inquiry screen in block B102, only user ID is input in block B110, whereby the program proceeds to block B108. From the user ID, such user attributes as shown in FIG. 7 can be extracted.

The application selection initial screen image includes a time schedule field and a management scenario field in the upper portion and the lower left portion of the screen, respectively. The same layout is employed in application selection screen images including the initial one. The time schedule field includes a horizontally extending time field (24 hours) area, an application icon, and buttons indicative of “save,” “read-in” and “online synchronization.” The application icon is displayed to cover a range corresponding to an execution time in the time field. Sign indicative of a present time is also displayed in the time field.

The management scenario field includes input boxes for inputting “execution time,” “measurement condition” and “application name,” and buttons indicative of “new reservation” and “save.”

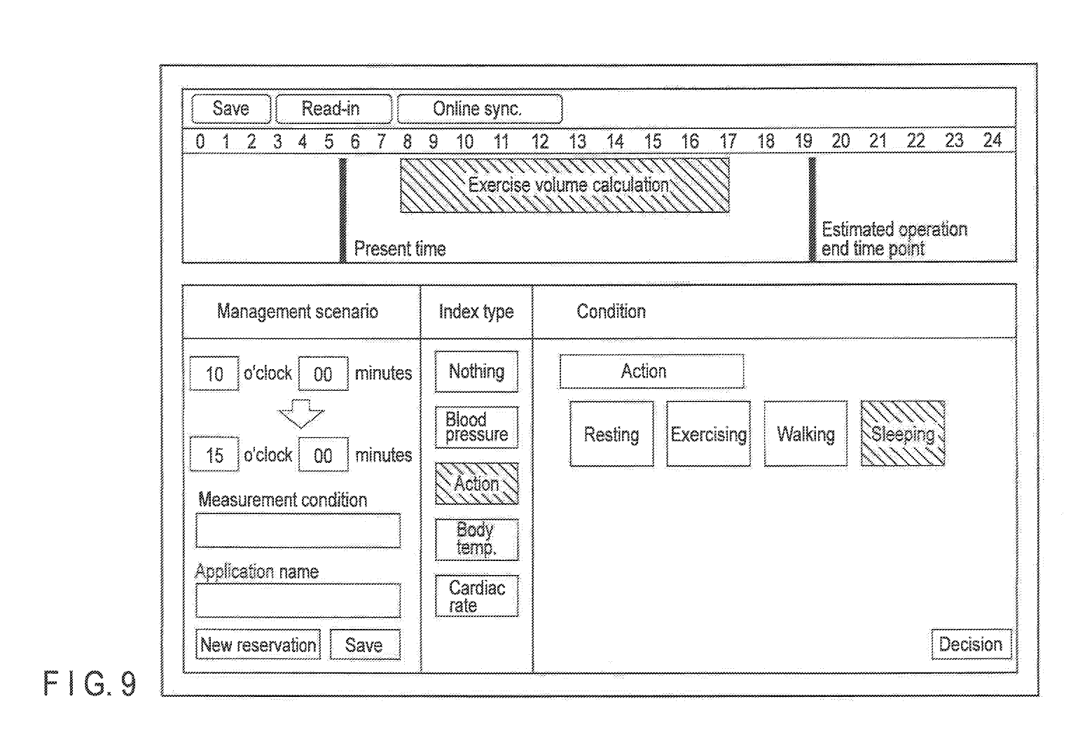

In the initial screen image, an application corresponding to the user ID and recommended to the user is selected from a large number of applications managed by the electronic device 12, and is displayed in the time schedule field. For instance, if scenarios corresponding to respective clinical histories are registered, an application recommended for high blood pressure is presented. In the case of FIG. 9, an exercise volume calculating application is recommended. The exercise volume can be measured using the acceleration sensor 32. Execution time periods are predetermined in accordance with respective recommended applications. Since exercise is often performed during a daytime, the execution time is set to a period from 8:00 to 17:00. However, the execution time can be changed. Namely, by dragging the left or right end of an icon indicating the activation start time or activation end time, the width (execution time period) of the icon indicative of the application can be changed. Further, when the icon of the application is double tapped, an execution time period, a measurement condition and an application name corresponding to the icon are displayed in the management scenario field. In this case, if the value(s) associated with, for example, the execution time period is changed, and then the “save” button is pressed, change of the execution time period is fixed.

In block B112, the user determines whether to register the recommended application in the scenario. If the user wish to register the recommended application, they tap the “save” button in the time schedule field. Unless the “save” button is tapped within a predetermined period after the start of display of FIG. 8, it is determined that the user does not wish to register the recommended application. If the “save” button is tapped within the predetermined period, the recommended application is saved in the scenario in block B114, and then the program proceeds to block B118. If the “save” button is not tapped within the predetermined period, the recommended application is canceled, and the program proceeds to block B118.

In blocks after block B118, processing of permitting the user to designate an application that they wish to execute using the biomedical sensor device 10, and to register the same. FIG. 9 shows an application registration screen image example displayed in block B118. More specifically, FIG. 9 shows an example where the recommended application is selected and registered in the scenario in block B112. When the recommended application has been selected, display of the icon is changed. Namely, before the selection, the background color is thin and/or drab, and/or characters are thin, for example. However, after the selection, the background color is thick and/or clean, and/or characters are thick, thereby emphasizing the icons. Thus, the states of the recommended application before and after the selection can be easily discriminated.

When the recommended application has been registered, the power consumption needed for the execution of the application can be estimated and the charge runout time of the rechargeable battery 42 can be estimated, an estimated operation end time is displayed in the time field of the time schedule field. In the example of FIG. 9, since the estimated operation end time (19:00) is later than an activation end time (17:00) of the exercise volume calculating application, there is no problem in the operation of the biomedical sensor device 10. However, in the opposite case, the execution time (start time and/or end time) of the recommended application must be modified, or the recommended application itself be canceled.

In the screen image of FIG. 9, when the execution of an application is newly registered on the screen image of FIG. 8, if the “measurement condition” box in the management scenario field is tapped, an index type area and a condition area are additionally shown on the right side of the management scenario field. The index type area is used to display candidates for the type of measurement condition, and includes, for example, “nothing in particular,” “blood pressure,” “action,” “body temperature” and “cardiac rate” buttons.

The condition area is used to display candidates for the measurement condition associated with the selected index type. If, for example, “action” has been selected as the index type, buttons of condition candidates associated with the action, such as “resting,” “exercising,” “walking” and “sleeping,” are displayed. As described above, each application is not merely activated at the activation start time, but is activated on condition that the living body is in a predetermined state. For instance, if “sleeping” has been selected as the condition, the corresponding application is activated only when the biomedical data indicates that the living body is during sleeping at the activation start time. If the biomedical data does not indicate that the living body is during sleeping at the activation start time, it is meaningless if a biomedical data value (s) is measured, and hence no application is activated. When the biomedical data has come to indicate “sleeping” within the execution time period, the application is activated. The action of the living body can be determined based on the outputs of the electrocardiograph 30, acceleration sensor 32, temperature sensor 24 and pulse wave meter 34. The sensors are not limited to the above-mentioned ones, but may also include other biomedical signal measuring sensors, such as a gyro sensor, a microphone and a blood oxygen sensor.

In the screen images shown in FIG. 9 et seq., when a button has been tapped, display is changed as well as the icon of the recommended application. Specifically, before selection, the background color and displayed characters are thin, for example. After the selection, the background color and characters become thick, the display form is changed, and the buttons are emphasized, for example. Thus, the states assumed before and after the selection are easily discriminated.

In block B118, the execution time period (i.e., the measuring time of a biomedical data value) of an application that the user wishes to register is input in the input boxes of “execution time period” in the management scenario field. For the input of time, a software keyboard may be displayed. to enable the user to input, a value in the input box. Alternatively, time options may be displayed to enable the user to select one of them, when the input box is tapped.

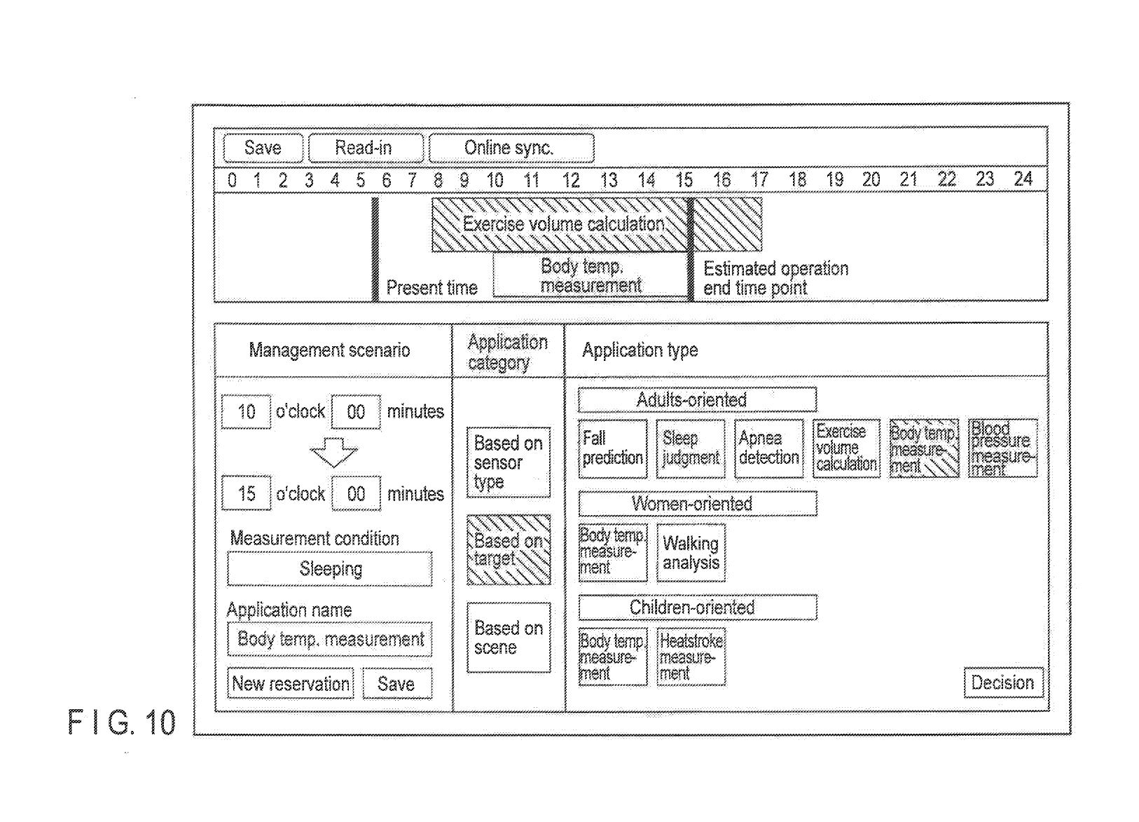

In block B120, the type of measurement condition is selected from the index type area. In block B122, conditions associated with the selected index type are displayed in the condition area. In block B124, one of the conditions is selected. In block B126, the screen image is shifted to that of FIG. 10. FIG. 10 shows a screen image assumed when “action” has been selected as the index type and “sleeping” has been selected as a condition associated with the action on the screen image of FIG. 9. Since “sleeping” has been selected as the measurement condition on the screen image of FIG. 9, “sleeping” is also input in the measurement control box in the management scenario field on the screen image of FIG. 10. On the screen image of FIG. 10, when “application name” in the management scenario field has been tapped, the application category area and an application type area are displayed instead of the index type area and the condition area on the screen image of FIG. 9, respectively. The application category area is used to display buttons for designating in which category, the application should be searched for. For instance, the buttons include “search based on sensor type,” “search based on target” and “search based on scene” buttons. When “search based on sensor type” has been selected, the application type area displays a group of candidates corresponding to applications performed using the electrocardiograph 30, a group of candidates corresponding to applications performed using the acceleration sensor 32, a group of candidates corresponding to applications performed using the temperature sensor 24, and a group of candidates corresponding to applications performed using the pulse wave meter 34. In contrast, when “search based on target” has been selected, the application type area displays groups of application candidates corresponding to respective user categories of the biomedical sensor device 10 (block B128). The target users are, for example, adults, women, children, etc. When “search based on scene” has been selected, the application type area displays application candidates corresponding to respective user environments of the biomedical sensor device 10, such as “sleeping,” “working,” “breaking time,” etc.

Since applications are thus displayed in association with respective categories, the user can easily detect and select a desired application. When an application has been selected from the application type area, the button corresponding thereto is emphasized, the selected application (in this example, temperature measurement) is input to the “application name” input box in the management scenario field, and an icon corresponding to the temperature measurement application is added in the time schedule field.

When an application to be registered in he management scenario field has been determined, power consumption needed to execute the application is estimated, the charge runout time of the rechargeable battery 42 is estimated, and the estimated operation end time point in the time field is changed to an earlier time point in block B130. Although the estimated operation end time point was 19:00 in FIG. 9, it is changed to 15:00 since the temperature measurement application has been added, as is shown in FIG. 10. Thus, it is evident that the exercise amount calculation application can be executed only until 15:00. Namely, it can be understood that the capacity of the rechargeable battery 42 is insufficient to execute all of the currently execution-scheduled scenario, and therefore that it is necessary to change the scenario. The change of scenario includes, for example, deletion of the application, and reduction of the execution time period of the application. In block B132, it is determined whether there is an instruction to change the scenario. The change instruction can be made by, for example, double tapping the management scenario field or an icon corresponding to an application to be changed. In block B134, the scenario is changed. If it has been determined in block B132 that there is no scenario change instruction, or if the scenario has been changed in block B134, the scenario is stored in the SSD 66 in block B136, and is sent to biomedical sensor device 10. The scenario sent to the biomedical sensor device 10 is stored in the flash memory 50. According to the circumstances, the scenario may be uploaded to the server 16 in block B136.

As described above, an application that the user wishes to make the biomedical sensor device 10 execute within the power supply capacity range of the rechargeable battery 42, and its execution time period, can be easily selected using the electronic device 12, and further a condition to be satisfied by biomedical data to activate the application can be defined, whereby the operation of the biomedical sensor device 10 can be optimized for individual users.

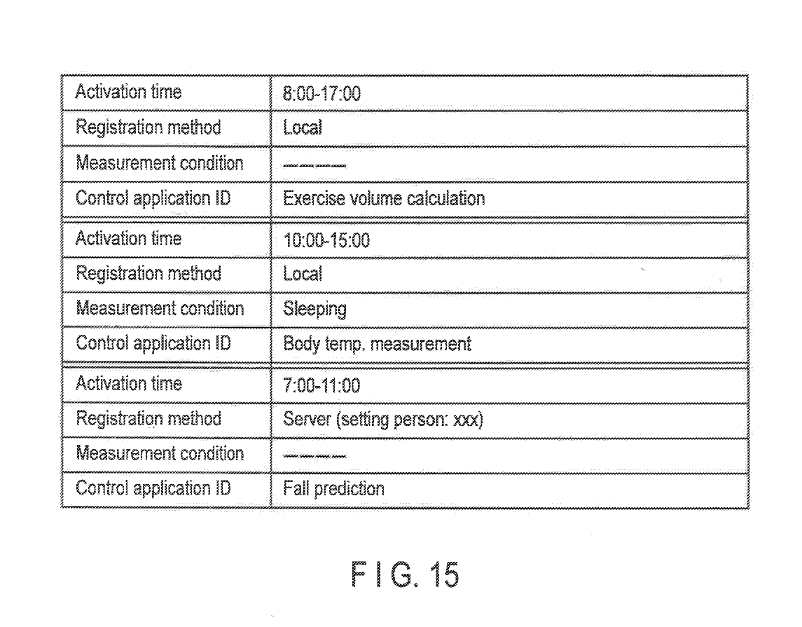

FIG. 15 shows a scenario example stored in the SSD 66 of the electronic device 12. As shown, in respective scenarios, sets, which. each include an execution time period, a registration method, a measurement condition; and a control application ID, are stored in association with applications to be activated. The registration method indicates the creator of a corresponding scenario. If the scenario creator is the user of the biomedical sensor device 10 (i.e., the user of the electronic device 12), the registration method is set local, while If the scenario is downloaded from the server 16, the registration method is set to the server 16 (setting person: xxx).

FIGS. 16 to 19 show formats of applications in detail. These formats are stored in the SSD 66 of the electronic device 12. Particular items of each application include application ID, the number of sensors used, the type(s) of sensors used, a temperature control method, an electrocardiogram. control method, an acceleration control method, a pulse wave control method, supposed users, supposed scenes of use, supposed. measurement conditions, a sensor control method for controlling a sensor whose normal operation. is not guaranteed, the amount of use of a microcomputer memory, the amount of calculation, a preceding determination result, etc. Data on “the type(s) of sensors used” is used to determine the application type when the application category “search based on sensor type” in FIG. 10 has been selected. Data on “supposed users” is used to determine the application type when the application category “search based on target” has been selected. Data on “supposed scenes of use” is used to determine the application type when the application category “search based on scenes” in FIG. 10 has been selected. Data on “supposed measurement conditions” is used to determine whether the application type is contradictory to the measurement condition(s). Data on “a sensor control method for controlling a sensor whose normal operation is not guaranteed” is used to determine whether the application can be selected. Data on the calculation amount is used to display the icon indicative of the application in a format corresponding to the calculation amount. Data on “a preceding determination result” is used to emphasize an application, lastly determined to be abnormal, in such application list as shown in FIG. 10.

FIG. 16 shows an example of an autonomic nerve analyzing application. In this case, the following definitions are made:

The number of sensor types=1;

Type of used sensor=electrocardiograph (electrocardiogram sensor);

Temperature control method=non-defined;

Electrocardiogram control method=non-defined;

Acceleration control method=sampling period of 32 msec;

Pulse wave control method=non-defined;

Supposed users=elderly adults, adults and women;

Supposed scenes of use=sleeping, working and resting;

Supposed measurement conditions=resting state, cardiac rate of 30 to 180;

Sensor control method for controlling a sensor whose normal operation is not guaranteed=use of sensor in synchronization with yy application, sampling period of Xx msec. or more;

Amount of use of microcomputer memory=16 k;

Amount of calculation=processing timing of 1 heartbeat, calculations of XX steps per 1 loop; and

Preceding determination result=abnormal.

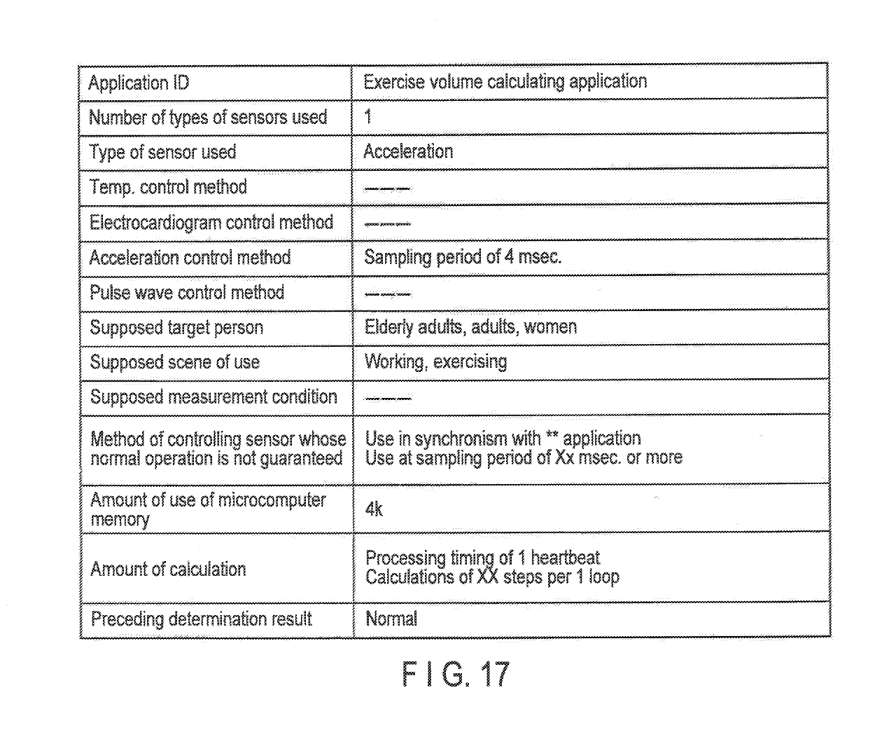

FIG. 17 shows an example of the exercise volume calculating application. In this case, the following definitions are made;

The number of sensor types=1;

Type of used sensor=acceleration;

Temperature control method=non-defined;

Electrocardiogram control method=non-defined;

Acceleration control method=sampling period of 4 msec.;

Pulse wave control method=non-defined;

Supposed users=elderly adults, adults and women;

Supposed scenes of use=working, exercising;

Supposed measurement conditions=non-defined;

Sensor control method for controlling a sensor whose normal operation is not guaranteed=use of sensor in synchronization with yy application, sampling period of Xx msec, or more;

Amount of use of microcomputer memory=4 k;

Amount of calculation=processing timing of 1 heartbeat, calculations of XX steps per 1 loop; and

Preceding determination result=normal.

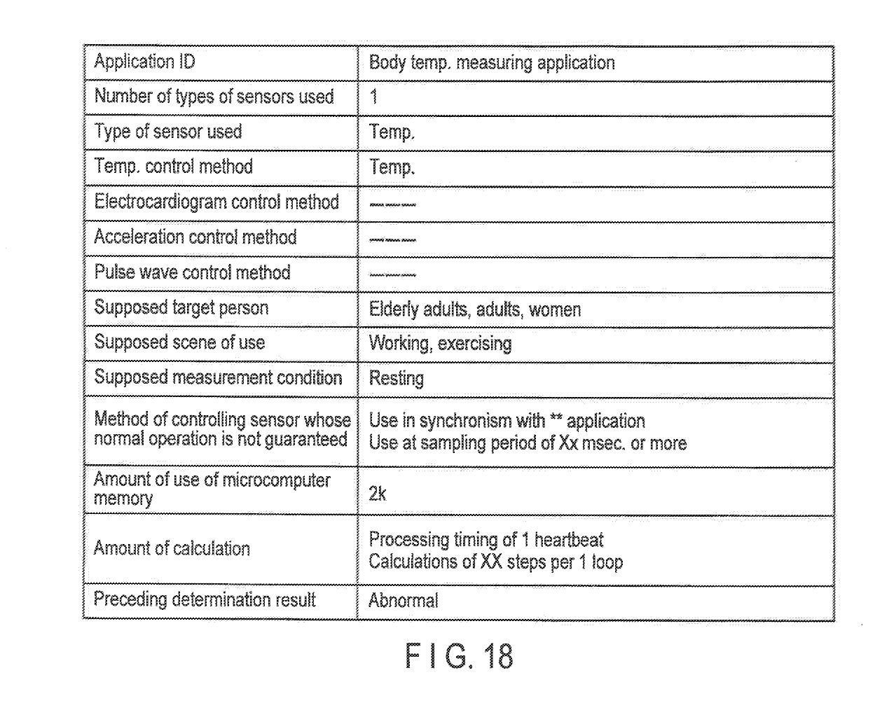

FIG. 18 shows an example of the temperature measuring application. In this case, the following definitions are made:

The number of sensor types=1;

Type of used sensor=temperature;

Temperature control method=temperature;

Electrocardiogram control method=non-defined;

Acceleration control method=non-defined;

Pulse wave control method=non-defined;

Supposed users=elderly adults, adults and women;

Supposed scenes of use=working, exercising;

Supposed measurement condition=resting;

Sensor control method for controlling a sensor whose normal operation is not guaranteed=use of sensor in synchronization with yy application, sampling period of Xx msec, or more;

Amount of use of microcomputer memory=2 k;

Amount of calculation=processing timing of 1 heartbeat, calculations of XX steps per 1 loop; and

Preceding determination result=normal,

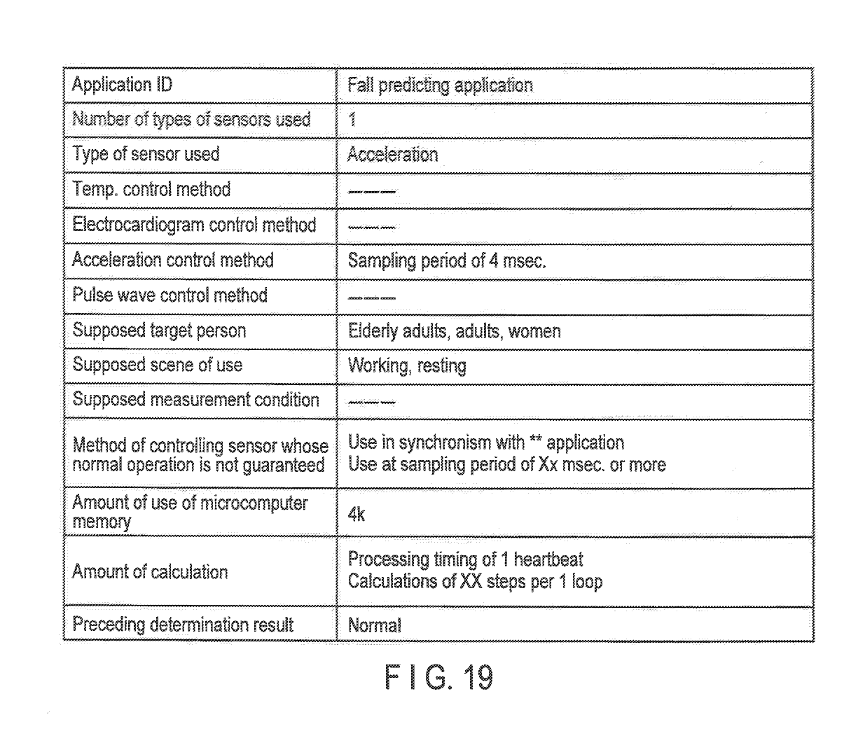

FIG. 19 shows an example of the failing prediction application in this case, the following definitions are made:

The number of sensor types=1;

Type of used sensor=acceleration;

Temperature control method=non-defined;

Electrocardiogram control method=non-defined;

Acceleration control method=sampling period of 4 msec.;

Pulse wave control method=non-defined;

Supposed users=elderly adults, adults;

Supposed scenes of use=working, resting;

Supposed measurement condition=non-defined;

Sensor control method for controlling a sensor whose normal operation is not guaranteed=use of sensor in synchronization with yy application, sampling period of Xx msec, or more;

Amount of use of microcomputer memory=4 k;

Amount of calculation=processing timing of heartbeat, calculations of XX steps per 1 loop; and

Preceding determination result=normal.

In the above description, the electrocardiograph, the acceleration sensor and the temperature sensor are used as examples of the sensors used. These sensors may be used individually or in a combination of two or three. Further, the aforementioned pulse wave meter may also be combined.

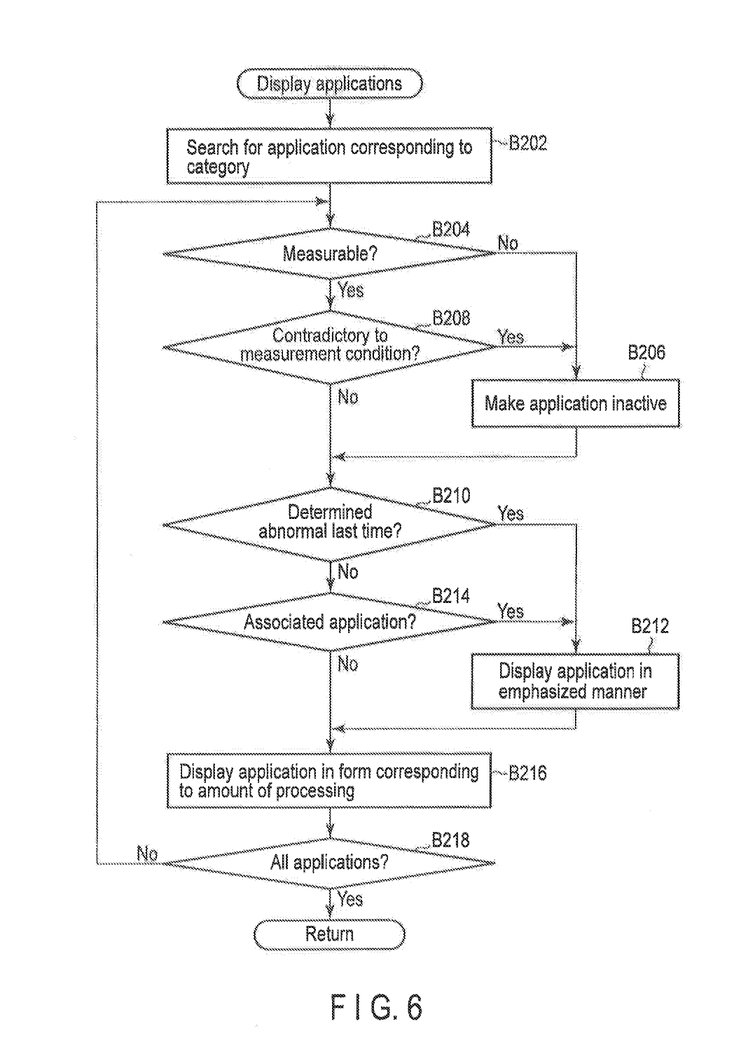

Referring then to FIG. 6, a detailed description will be given of the application display block B128 shown in FIG. 5.

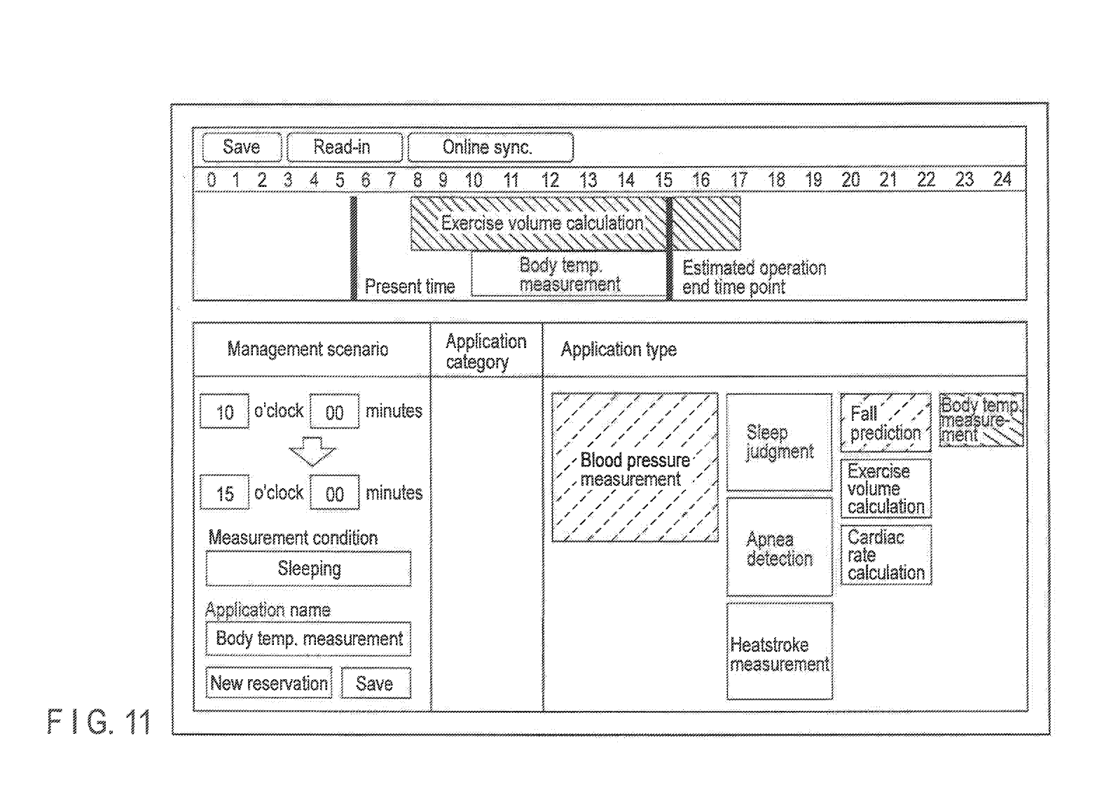

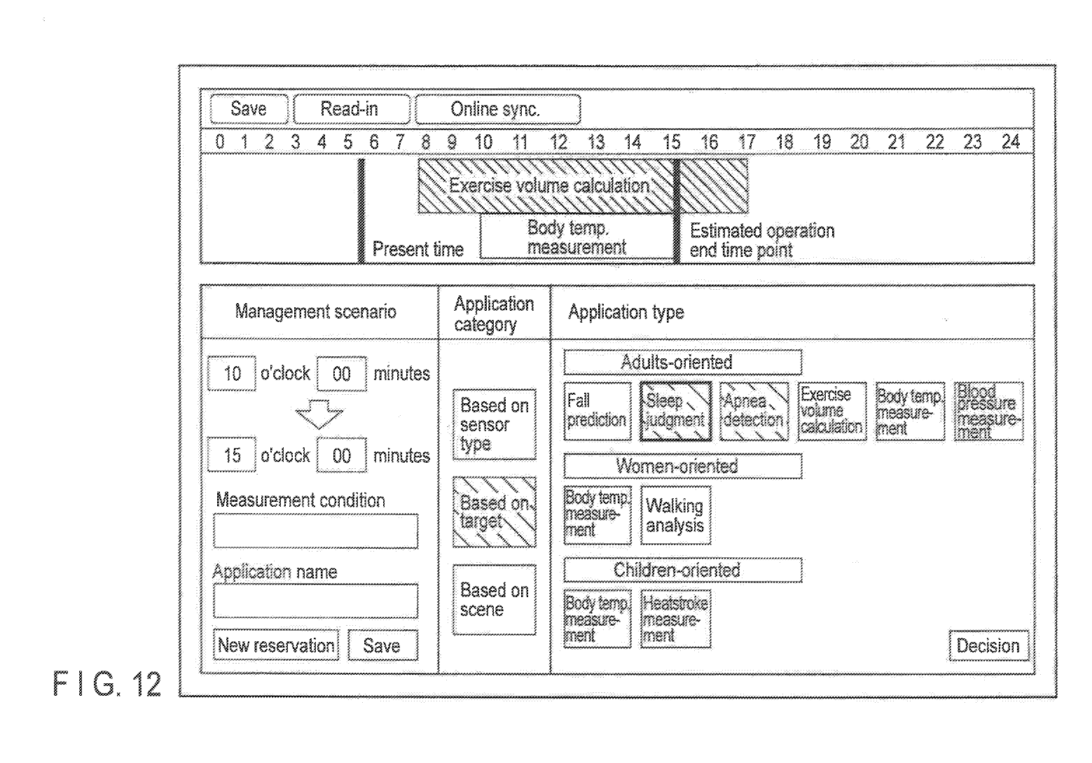

When applications classified in accordance with types corresponding to application categories are displayed as shown in FIG. 10 (block B202), it is determined in block B204 whether a biomedical data value as a measurement target of an application can be measured by the biomedical sensor device 10. If it is determined that the biomedical data value cannot be measured, the program proceeds to block B206, where a display button corresponding to the application is displayed in a non-emphasized manner so as not to be touched, and is made inactive so as not to make a decision even if it is selected. For instance, blood pressure may be hard to measure depending upon the type or model of a biomedical sensor device, because a large amount of arithmetic throughput is required for measuring the same. In this case, a “blood pressure measurement” button is made inactive, and its background color and/or its characters are made thin so that the button can be easily understood to be inactive. Broken hatching made on the “blood pressure measurement” button in FIG. 11 means a non-emphasized display. The biomedical sensor devices 10 that can be used in the health management system are of various models, and may be able to measure different types of biomedical data. Further, model information associated with the biomedical sensor devices 10 may be registered when new user registration shown in FIG. 7 is performed.

If it is difficult for the biomedical sensor device 10 currently attached to a human body to measure blood pressure, a “blood pressure” button in the index type area of FIG. 9 may also be displayed in a non-emphasized manner.

If it is determined in block B204 that the biomedical data value can be measured, it is determined. in block B208 whether a biomedical data value as the measurement target of an application is contradictory to a measurement condition. This determination is performed by comparing a supposed measurement condition shown in the application particulars of FIG. 16 with an actually set measurement condition. If the measurement target is contradictory to the measurement condition, in block B206, the display button of the application is displayed in a non-emphasized manner so as not to be touched and is made inactive so as not to make a decision even if it is selected. For instance, walking analysis regards a living body's walking state as a supposed measurement condition. Therefore, if the measurement condition is “sleeping” as in FIG. 10, the display button cannot be selected. As a result, the “walking analysis” button in FIG. 10 should be displayed in a non-emphasized manner using broken hatching, and be made inactive.

If it is determined that the measurement target is not contradictory to the measurement condition in block B204, and after the button of the application is changed to a non-emphasized display in block B206, the program proceeds to block B210, where it is determined whether a preceding determination result associated with the biomedical data value measured by the application is abnormal (see FIG. 16). If it is determined that the preceding determination result is abnormal, the program proceeds to block B212, where the application is displayed in an emphasized manner like a “sleep determination” application in the application list of FIG. 12. The preceding determination result “abnormal” means that it is preferable to continuously monitor the biomedical data, and therefore its display form is changed to stimulate user selection. Regarding an application whose preceding determination result is normal, it is determined in block B214 whether this application is associated with the application whose determination result is abnormal. If it is determined that the applications are associated with each other, the application whose preceding determination result was normal is displayed in block B212 in an emphasized manner like an “apnea detection” application in the application list of FIG. 12. The associated applications are associated with each other in biomedical data values to be measured, and hence it is preferable to also continuously monitor the biomedical data whose preceding determination result was abnormal. Therefore, the display forms of the two applications are changed to stimulate user selection.

In contrast, if it is determined that the application is not associated with the application whose preceding determination result was abnormal, and after the button of the application is changed to a non-emphasized display in block B212, the button of the application is changed in block B216 to a size corresponding to the amount of calculation (see FIG. 16) for executing the application. FIG. 11 shows a display example in block B216. Based on the size of the application button, the user can select a to-be-registered application considering the processing performance of the biomedical sensor device 10.

In block B218, it is determined whether all applications have been processed. If the answer in block B218 is No, processings in block B204 et seq. are repeated.

The above description relates to the case where the user creates a scenario. However, a scenario may be downloaded from server 16.

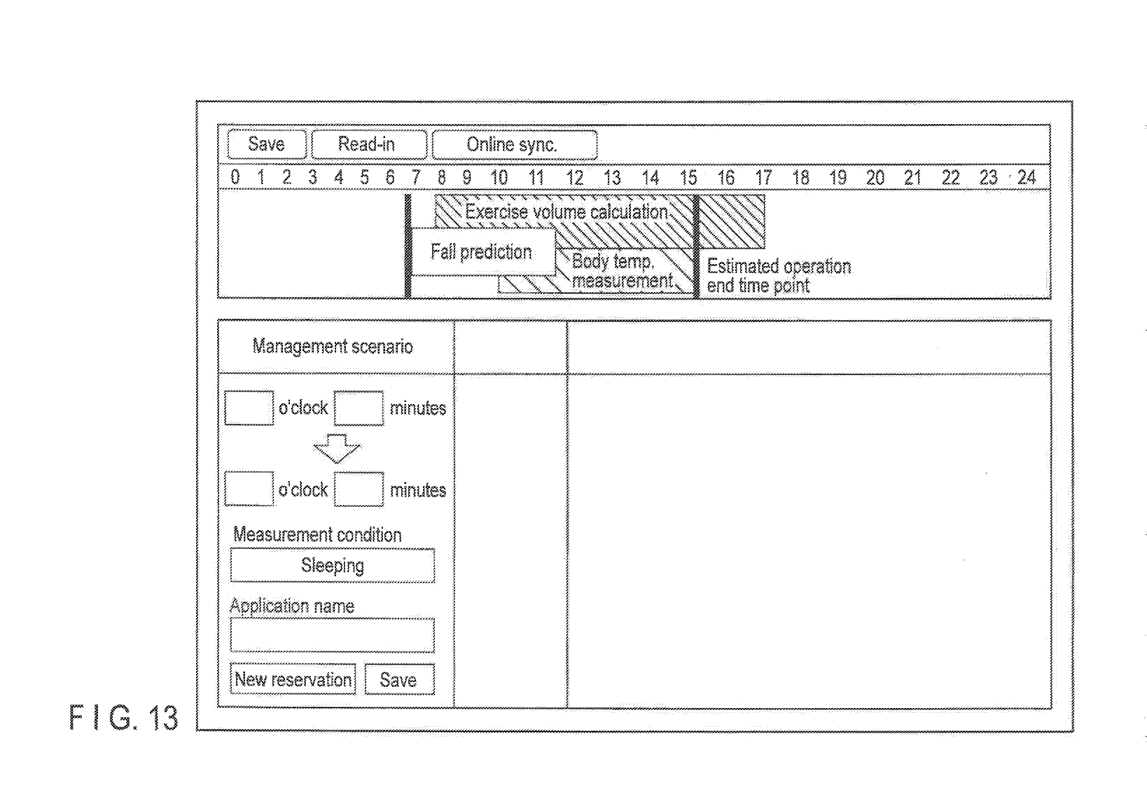

For instance, if an “online synchronization” button in the time schedule field is tapped after the exercise volume calculating application and the body temperature measuring application scenarios are registered, as is shown in FIG. 10, a “fall prediction” application is downloaded from the server 16 and a “fall prediction” button is displayed in the time schedule field, as is shown in FIG. 13, The “fall prediction” application may be set in the biomedical sensor device 10 attached to users by a medical agency, an enterprise health management association, etc., as the operator of the health management system. As shown in FIG. 13, the buttons of the applications registered by the user are displayed in a different form (in, for example, a different color) from the application button downloaded from server 16. This enables the user to easily determine whether each of scenarios simultaneously set in the biomedical sensor device 10 is registered by the user or server 16.

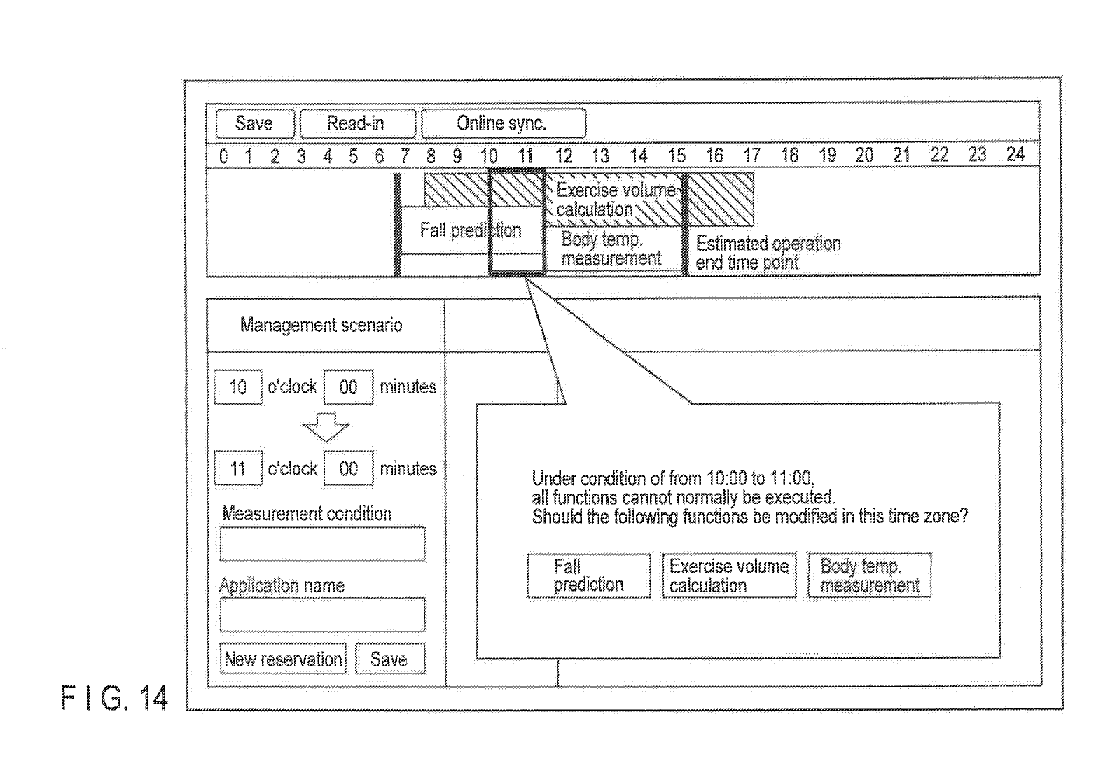

In the scenario of the “fall prediction” application, the application is scheduled to be executed from 7:00 to 11:00. Accordingly, during the period from 10:00 to 11:00, the “fall prediction” application is executed simultaneously with the “exercise volume calculation” and “body temperature measurement” applications already registered. If the total amount of calculation of the three applications exceeds the processing capacity of the CPU 3 of the biomedical sensor device 10, such en alert window as shown in FIG. 14 is displayed. From this, the user understands that the three applications cannot simultaneously be executed from 10:00 to 11:00. In accordance with the alert message, the user selects an application for stopping the execution from 10:00 to 11:00.

As described above, in the embodiment, when an application to be executed by the biomedical sensor device 10 is registered, its execution time period, and an activation condition associated with biomedical data output from the biomedical sensor device 10, can be simultaneously registered. This enables the biomedical sensor device 10 to be appropriately customized in accordance with the behavior of the user and situations.

Further, since each registered application is displayed in the time field in the form of an icon having a size corresponding to the execution time period, a plurality of applications can be registered with high operability.

Further, the biomedical sensor device 10 is powered by a rechargeable battery. If applications to be executed. are increased, the power of the battery is reduced. By displaying, on the application registration screen image, the operation end time of the biomedical sensor device 10 estimated from power reduction of the rechargeable battery 42, the impossibility of execution of an application due the charge runout of the battery can be detected in advance, thereby enhancing the convenience of the scenario registration.

Since application option icons are displayed with being classified in accordance with categories selected by the user at the time of the application registration, an application can be easily selected.

When application options are displayed, if an execution condition supposed for an application is contradictory to an activation condition, an icon corresponding to the application is displayed so that the contradiction is known from the icon. As a result, an appropriate application can be easily selected.

Further, when application options are displayed, icons indicative of the applications are displayed so that the applications that do not guarantee a normal operation of the biomedical sensor device when they are executed, can be identified. Accordingly, the operation of the sensor device can be controlled to perform measurement with a combination of applications that guarantees normal operation of the sensor device.

Yet further, when application options are displayed, icons indicative of the, applications are displayed with sizes corresponding to calculation amounts for executing the respective applications. This prevents selection of a number of applications that exceeds the processing capacity of biomedical sensor device 10. In other words, appropriate applications can be easily selected.

In addition, application options are displayed so that an application(s) associated with biomedical data that was determined abnormal in a preceding determination, or an application(s) associated with the first-mentioned application, can be identified. As a result, a biomedical data value (s) that may preferably be measured at this time can be recognized, and hence an appropriate application(s) can be selected.

Since the processing of the embodiment may be executed by a computer program, the same advantage as the embodiment can be easily realized simply by installing the computer program in a computer through a computer-readable recording medium storing the program.

While certain embodiments have been described, these embodiments have been presented by way of example only and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic device configured to control a sensor device executing applications to measure different biomedical data values, the electronic device comprising:
a first display controller to display a first image for designating an application to be executed by the sensor device, designating an execution time of the application, and designating an activation condition associated with a biomedical data value measured by the sensor device; and
a transmitter to transmit, to the sensor device, the designated application, the designated execution time and the designated activation condition.

2. The electronic device of claim 1, further comprising a second display controller to display a second image for displaying the designated application and the designated execution time along a time axis.

3. The electronic device of claim 2, wherein the second display controller displays the second image on a screen on which the first image is displayed.

4. electronic device of claim 2, wherein
the sensor device is powered by a rechargeable battery; and
the second image includes an estimated operation end time point of the sensor device.

5. The electronic device of claim 4, wherein the estimated operation end time point in the second image is updated in accordance with a change in the designated application and the designated execution time.

6. The electronic device of claim 1, wherein the first image includes application options in association with types of sensor units in the sensor device, with user categories of the sensor device, or with application execution scenes.

7. The electronic, device of claim 6, wherein the sensor device comprises an electrocardiograph, a pulse wave meter, an acceleration sensor, and a temperature sensor.

8. The electronic device of claim 1, wherein the first, image includes an application option in a predetermined manner when a condition for executing the application contradicts to the designated activation condition.

9. The electronic device of claim 1, wherein the first image includes an application option in a predetermined manner when a normal operation of the sensor device is not guaranteed by an execution of the application.

10. The electronic device of claim 9, further comprising a third display controller to display a third image for indicating that the normal operation of the sensor device is not guaranteed by the execution of the application.

11. The electronic device of claim 1, wherein the first image includes an application option in a predetermined manner indicating an amount of processing of the application.

12. The electronic device of claim 1, wherein the first image includes an application option of a first application in a predetermined manner when a biomedical data value measured when the first application was executed was abnormal.

13. The electronic device of claim 12, wherein the first image includes an application option of a second application in a predetermined manner when a biomedical, data value measured when the first application was executed was abnormal.

14. The electronic device of claim 2, further comprising a receiver to receive an application, an execution time of the application, and an activation condition from a server, and
wherein the second image includes an first application icon of a first application designated by a user of the electronic device in a first manner, and a second application icon of a second application received from the server in a second manner.

15. A method of controlling a sensor device executing applications to measure different biomedical data values, the method comprising:
displaying a first image for designating an application to be executed by the sensor device, designating an execution time of the application, and designating an activation condition associated with a biomedical data value measured by the sensor device; and
transmitting, to the sensor device, the designated application, the designated execution time and the designated activation condition.

16. The method of claim 15, further comprising:
displaying a second image for displaying the designated application. and the designated. execution time along a time axis.

17. The method of claim 16, further comprising
displaying an estimated operation end time point of the sensor device in the second image.

18. A non-transitory computer-readable storage medium storing computer-executable instructions that, when executed, cause a computer to:
display a first image for designating an application to be executed by a sensor device, designating an execution time of the application, and designating an activation condition associated with a biomedical data value measured hr the sensor device; and
transmit, to the sensor device, the designated application, the designated execution time and the designated activation condition.

19. The storage medium of claim 18, wherein the instructions further cause a computer to:
display a second image for displaying the designated application and the designated execution time along a time axis.

20. The method of claim 16, wherein the instructions further cause a computer to
display an estimated operation end time of the sensor device in the second image.
